(19) United States
(12) Patent Application Publication
(10) Pub. No.: US 2005/0038063 A1
(43) Pub. Date: Feb. 17, 2005

(54) Method of treating acute pain with unitary dosage form comprising ibuprofen and oxycodone

(76) Inventors: Kenneth Newman, Katonah, NY (US); Wattanaporn Abramowitz, West Windsor, NJ (US); Andreas Grill, Hauppauge, NY (US); Pablo Davila-Zavala, East Windsor, NJ (US); Fuxing Tang, Deer Park, NY (US)

Correspondence Address: DARBY & DARBY P.C., P. O. BOX 5257, NEW YORK, NY, 10150-5257, US

(21) Appl. No.: 10/861,239

(22) Filed: Jun. 2, 2004

Related U.S. Application Data
(63) Continuation-in-part of application No. 10/725,246, filed on Dec. 1, 2003.
(60) Provisional application No. 60/429,944, filed on Nov. 29, 2002.
(60) Provisional application No. 60/453,044, filed on Mar. 7, 2003.
(60) Provisional application No. 60/506,632, filed on Sep. 26, 2003.

(51) Int. Cl. A61K 31/485; A61K 31/192; G06F 17/60

(52) U.S. Cl. 514/282; 514/570; 705/2

(57) ABSTRACT

The present invention is a method of achieving fast onset of pain relief for acute pain in a patient in need thereof comprising orally administering a unitary formulation (or oral dosage form) containing an effective analgesic amount of (a) oxycodone or a pharmaceutically acceptable salt thereof and (b) ibuprofen or a pharmaceutically acceptable salt thereof. Preferably, the unitary formulation contains (a) oxycodone or a pharmaceutically acceptable salt thereof and (b) ibuprofen or a pharmaceutically acceptable salt thereof at a weight ratio of from about 1:20 to about 1:100 and more preferably about 1:40 to about 1:80, based on the weights of molar equivalents of oxycodone hydrochloride and ibuprofen, respectively. Preferably, an amount of oxycodone and ibuprofen effective to provide partial or complete pain relief within 30 minutes is administered. More preferably, the amount is sufficient to provide partial or complete pain relief within 25 minutes. It has been discovered that administration of an oral dosage form containing both oxycodone and ibuprofen provides earlier onset of pain relief than administration of either active ingredient alone. Moreover, the earlier onset of pain relief may be attributable at least in part to administration of a single dosage form containing both active ingredients as opposed to administering oxycodone and ibuprofen in separate oral dosage forms (i.e., administration of a first dosage form containing oxycodone and a second dosage form containing ibuprofen). The method of the present invention is particularly useful for treating acute postoperative pain, including, but not limited to, moderate and/or severe acute postoperative pain (such as that resulting from dental surgery).

This application is a continuation-in-part of U.S. Ser. No. 10/725,246, filed Dec. 1, 2003, which claims the benefit of U.S. Provisional Patent Application No. 60,429,944, filed Nov. 29, 2002, U.S. Provisional Patent Application No. 60/453,044, filed Mar. 7, 2003, and U.S. Provisional Patent Application No. 60/506,632, filed Sep. 26, 2003, all of which are hereby incorporated by reference.

### FIELD OF THE INVENTION

The present invention relates to a method of treating acute pain (e.g., acute postoperative pain) by administering a composition comprising ibuprofen and oxycodone, whereby a faster onset of pain relief is achieved.

### BACKGROUND OF THE INVENTION

Oral analgesics, such as ibuprofen (U.S. Pat. Nos. 3,228,831 and 3,385,886), and narcotic analgesics (e.g., oxycodone), have been known for decades. Narcotic analgesics, however, can be addictive and subjected to abuse by parenteral administration. As a result, there has been research in reducing the dosage of narcotic analgesics necessary to obtain pain relief. For example, U.S. Pat. No. 4,569,937 discloses an analgesic pharmaceutical composition containing a synergistic effective amount of oxycodone and ibuprofen.

Oral analgesics are not typically administered for moderate and severe acute pain when fast pain relief is a primary goal. As noted in Basics of Anesthesia, 4^{th} Ed., R. K. Stoelting and R. D. Miller (2000), p. 428:
“Oral administration of analgesics is not considered optimal for management of moderate to severe acute postoperative pain, principally because of the lack of titratability and prolonged time to peak effect. Traditionally, postoperative patients are switched [from parenteral analgesics] to oral analgesics (aspirin, acetaminophen, NSAIDs) when pain has diminished to the extent that the need for rapid adjustments in the level of analgesia is unlikely . . . . [T]here is a growing need for oral analgesics that are efficacious in the treatment of moderate to severe acute postoperative pain.”

Cooper et al., Clinical Pharmacology & Therapeutics, PII-9 (February 1993), report the results of a clinical study where (1) 2×®mg ibuprofen capsules with a 5 mg oxycodone capsule, (2) 2×200 mg ibuprofen capsules and a placebo capsule, or (3) 3 placebo capsules were administered to patients having pain due to surgical removal of impacted teeth. See also Dionne, J. Oral Maxillofac Surg., 57:673-678 (1999).

There is a need for an oral analgesic which provides fast pain relief.

### SUMMARY OF THE INVENTION

The present invention is a method of achieving fast onset of pain relief for acute pain in a patient in need thereof comprising orally administering a unitary formulation (or oral dosage form) containing an effective analgesic amount of (a) oxycodone or a pharmaceutically acceptable salt thereof and (b) ibuprofen or a pharmaceutically acceptable salt thereof. Preferably, the unitary formulation contains (a) oxycodone or a pharmaceutically acceptable salt thereof and (b) ibuprofen or a pharmaceutically acceptable salt thereof at a weight ratio of from about 1:20 (based on the weight of a molar equivalent of oxycodone hydrochloride and the free acid of ibuprofen, respectively) to about 1:100 and more preferably about 1:40 to about 1:80. Preferably, an amount of oxycodone and ibuprofen effective to provide partial or complete pain relief within 30 minutes is administered. More preferably, the amount is sufficient to provide partial or complete pain relief within 25 minutes. It has been discovered that administration of an oral dosage form containing both oxycodone and ibuprofen provides earlier onset of pain relief than administration of either active ingredient alone. Moreover, the earlier onset of pain relief may be attributable at least in part to administration of a single dosage form containing both active ingredients as opposed to administering oxycodone and ibuprofen in separate oral dosage forms (i.e., administration of a first dosage form containing oxycodone and a second dosage form containing ibuprofen). The method of the present invention is particularly useful for treating acute postoperative pain, including, but not limited to, moderate and/or severe acute postoperative pain (such as that resulting from dental surgery).

According to one preferred embodiment, the oral dosage form comprises from about 5 to about 10 mg of oxycodone or a pharmaceutically acceptable salt thereof (based on the weight of a molar equivalent of oxycodone hydrochloride and the free acid of ibuprofen, respectively) and from about 350 to about 500 mg of ibuprofen or a pharmaceutically acceptable salt thereof. For example, the oral dosage form may comprise about 5 mg of oxycodone or a pharmaceutically acceptable salt thereof (such as oxycodone HCl) and about 400 mg of ibuprofen or a pharmaceutically acceptable salt thereof. Another example is an oral dosage form which comprises about 10 mg of oxycodone or a pharmaceutically acceptable salt thereof (such as oxycodone HCl) and about 400 mg of ibuprofen or a pharmaceutically acceptable salt thereof.

The present invention also provides a method of treating acute pain in a patient in need thereof by orally administering an oral dosage form comprising from about 5 to about 10 mg of oxycodone or a pharmaceutically acceptable salt thereof and from about 350 to about 500 mg of ibuprofen or a pharmaceutically acceptable salt thereof. According to a preferred embodiment, the oral dosage form comprises about 5 or about 10 mg of oxycodone or a pharmaceutically acceptable salt thereof (such as oxycodone HCl) and about 400 mg of ibuprofen.

Yet another embodiment is a method for accelerating onset of pain relief in acute postoperative pain experienced by a patient post-anesthesia by administering to the patient an oral dosage form comprising (a) ibuprofen or a pharmaceutically acceptable salt thereof and (b) oxycodone or a pharmaceutically acceptable salt thereof (such as oxycodone HCl), at a weight ratio within the range of 20:1 to 100:1. Preferably, the weight ratio ranges from about 40:1 to about 80:1. The oral dosage form contains from about 5 to about 10 mg of oxycodone or a pharmaceutically acceptable salt thereof. The term “post-anesthesia” refers to a patient previously anaesthetized who is suffering from pain after the anesthesia partially or completely fades or wears off.

Unexpectedly, treatment of acute pain according to the present invention, i.e., administering to a subject experiencing such pain a unitary dosage form containing oxycodone and ibuprofen, results in a statistically significant earlier onset of pain relief than administration of either ingredient alone. A single dosage form has been shown to have a different (faster) ibuprofen pharmacokinetic profile, which is consistent with a significantly earlier onset of pain relief. See FIG. 4 and Example 8 wherein the maximum ibuprofen plasma concentration with the unitary dosage form is achieved earlier as compared to the two dosage form combination. Furthermore, a single dosage form has been shown to have a faster oxycodone dissolution rate and result in more rapid absorption of oxycodone. See FIGS. 12 and 13 (30-60 minutes) and Example 10.

The unitary dosage form of the present invention also permits the use of higher amounts of ibuprofen in the dosage form without a deterrent increase of the side-effects attendant to administration of this analgesic.

Yet another embodiment is a method of treating a patient suffering from acute pain comprising the steps of:
(a) marketing to doctors an ibuprofen/oxycodone unitary oral dosage form comprising from about 5 to about 10 mg oxycodone or a pharmaceutiaclly acceptable salt thereof (e.g., about 5 or 10 mg oxycodone hydrochloride) and from about 350 to about 500 mg ibuprofen or a pharmaceutically acceptable salt thereof (e.g., about 400 mg ibuprofen) for the treatment of acute pain and as:
   (i) having a faster onset of pain relief than contemporaneous administration of the oxycodone and ibuprofen in separate oral dosage forms,
   (ii) exhibiting a more rapid absorption of the oxycodone than contemporaneous administration of the oxycodone and ibuprofen in separate oral dosage forms, and/or
   (iii) providing partial or complete pain relief within 25 minutes after administration;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient. Optionally, the ibuprofen/oxycodone unitary oral dosage form is marketed for the treatment of moderate, severe, or moderate to severe acute pain, or acute postoperative pain.

Yet another embodiment is a method for treating a patient suffering from acute pain comprising the steps of:
(a) providing to the marketplace an ibuprofen/oxycodone unitary oral dosage form comprising from about 5 to about 10 mg oxycodone or a pharmaceutiaclly acceptable salt thereof (e.g., about 5 or 10 mg oxycodone hydrochloride) and from about 350 to about 500 mg ibuprofen or a pharmaceutically acceptable salt thereof (e.g., about 400 mg ibuprofen) for the treatment of acute pain;
(b) sensitizing a doctor to a product claim that the unitary oral dosage form:
   (i) has a faster onset of pain relief than contemporaneous administration of the oxycodone and ibuprofen in separate oral dosage forms,
   (ii) exhibits a more rapid absorption of the oxycodone than contemporaneous administration of the oxycodone and ibuprofen in separate oral dosage forms, and/or
   (iii) provides partial or complete pain relief within 25 minutes after administration;
(c) diagnosing a patient as suffering from acute pain by the doctor;
(d) selectively prescribing the ibuprofen/oxycodone unitary oral dosage form to the patient by the sensitized doctor in view of the diagnosis of the patient; and
(e) orally administering the prescribed ibuprofen/oxycodone unitary oral dosage form to the patient. Optionally, the ibuprofen/oxycodone unitary oral dosage form is marketed for the treatment of moderate, severe, or moderate to severe acute pain, or acute postoperative pain.

Yet another embodiment is a method for treating a patient suffering from acute pain comprising the steps of:
(a) purchasing an ibuprofen/oxycodone unitary oral dosage form comprising from about 5 to about 10 mg oxycodone or a pharmaceutiaclly acceptable salt thereof (e.g., about 5 or 10 mg oxycodone hydrochloride) and from about 350 to about 500 mg ibuprofen or a pharmaceutically acceptable salt thereof (e.g., about 400 mg ibuprofen);
(b) tendering a prescription for the patient suffering from acute pain before completing the purchase transaction, the prescription having been received in response to a diagnosis of acute pain by a doctor, the doctor
   (1) having been sensitized to a product claim that the unitary oral dosage form:
      (i) has a faster onset of pain relief than contemporaneous administration of the oxycodone and ibuprofen in separate oral dosage forms,
      (ii) exhibits a more rapid absorption of the oxycodone than contemporaneous administration of the oxycodone and ibuprofen in separate oral dosage forms, and/or
      (iii) provides partial or complete pain relief within 25 minutes after administration; and
   (2) having selectively prescribed the unitary dosage form; and
(c) orally administering the purchased ibuprofen/oxycodone unitary oral dosage form to the patient.

Yet another embodiment is a unitary dosage form comprising (a) oxycodone or a pharmaceutically acceptable salt thereof, (b) ibuprofen or a pharmaceutically acceptable salt thereof, and (c) an anti-picking effective amount of silicified microcrystalline cellulose. The unitary dosage form may be prepared by direct compression or wet granulation. The tablet preferably has a hardness of from about 12 to about 18 kp.

A preferred directly compressed unitary dosage form of the present invention comprises (a) from about 0.7 to about 1.7% by weight of oxycodone or a pharmaceutically acceptable salt thereof (based on the weight of a molar equivalent of oxycodone hydrochloride), (b) from about 64 to about 77% by weight of ibuprofen or a pharmaceutically acceptable salt thereof (based on the weight of a molar equivalent of the free acid of ibuprofen), and (c) from about 15 to about 22% by weight of silicified microcrystalline cellulose, based upon 100% total weight of the directly compressed unitary dosage form.

### BRIEF DESCRIPTION OF THE DRAWINGS

FIGS. 1-3 show the pain intensity difference (PID), pain relief (PR) scores, and combined pain relief and pain intensity difference (PRID), respectively, over 6 hours for the pooled data from the two clinical studies described in Example 7 for 5 mg oxycodone HCl/400 mg ibuprofen, 400 mg ibuprofen, 5 mg oxycodone HCl, and placebo.

FIG. 4 shows a graph of the ibuprofen plasma concentration (μg/mL) versus time (hours) after administration of (1) a 5 mg oxycodone HCl/400 mg ibuprofen tablet and (2) a 5 mg oxycodone HCl tablet with 2×200 mg ibuprofen tablets in Example 8.

FIG. 5 shows a graph of the oxycodone plasma concentration (μg/mL) versus time (hours) after administration of (1) a 5 mg oxycodone HCl/400 mg ibuprofen tablet and (2) a 5 mg oxycodone HCl tablet with 2×200 mg ibuprofen tablets in Example 8.

FIG. 6 is a bar graph showing the effects of increasing concentrations of ibuprofen on the permeability (Papp) of oxycodone across Caco-2 cell monolayers. The asterisks (*) indicates a significance level of p<0.05, when compared with the permeability value in the absence of ibuprofen.

FIG. 7 is a bar graph showing the effects of increasing concentrations of ibuprofen on the amount of oxycodone transported across Caco-2 cell monolayers after the initial 20 minute-transport period. The asterisks (*) indicates a significance level of p<0.05, when compared with the permeability value in the absence of ibuprofen.

FIG. 8 is a bar graph showing the effects of increasing concentrations of oxycodone on the permeability (Papp) of ibuprofen across Caco-2 cell monolayers.

FIG. 9 is a schematic of the continuous dissolution/Caco-2 system described in Example 10.

FIG. 10 is a graph of the percentage by weight of ibuprofen dissolved (mean±standard deviation, n=3) over 60 minutes from a 400 mg ibuprofen/5 mg oxycodone hydrochloride tablet (♦), 2 Nuprin® tablets (200 mg ibuprofen per tablet) (▪), and the combination of 2 Nuprin® tablets (200 mg ibuprofen per tablet) and 1 Roxicodone™ tablet (5 mg oxycodone hydrochloride) (▴) in fasted state simulated intestinal fluid (FaSSIF) buffer as determined by the dissolution procedure described in Example 10.

FIG. 11 is a graph of the percentage by weight of ibuprofen absorbed (mean±standard deviation, n=3) over 60 minutes from a 400 mg ibuprofen/5 mg oxycodone tablet (♦), 2 Nuprin® tablets (200 mg ibuprofen per tablet) (▪), and the combination of 2 Nuprin® tablets (200 mg ibuprofen per tablet) and 1 Roxicodone™ tablet (5 mg oxycodone hydrochloride) (▴) in FaSSIF buffer as determined by the dissolution procedure described in Example 10.

FIG. 12 is a graph of the percentage by weight of oxycodone dissolved (mean±standard deviation, n=3) over 60 minutes from 1 tablet of 400 mg ibuprofen/5 mg oxycodone hydrochloride (♦), 1 Roxicodone™ tablet (5 mg oxycodone hydrochloride) (▪), and the combination of 2 Nuprin® tablets (200 mg ibuprofen per tablet) and 1 Roxicodone™ tablet (5 mg oxycodone hydrochloride) (▴) in FaSSIF buffer as determined by the dissolution procedure described in Example 10.

FIG. 13 is a graph of the percentage by weight of oxycodone absorbed (mean±standard deviation, n=3) over 60 minutes from 1 tablet of 400 mg ibuprofen/5 mg oxycodone hydrochloride (♦), 1 Roxicodone™ tablet (5 mg oxycodone hydrochloride) (▪), and the combination of 2 Nuprin® tablets (200 mg ibuprofen per tablet) and 1 Roxicodone® tablet (5 mg oxycodone hydrochloride) (▴) in FaSSIF buffer as determined by the dissolution procedure described in Example 10.

### DETAILED DESCRIPTION OF THE INVENTION

As used herein, the term “about” means within 10% of a given value, preferably within 5%, and more preferably within 1% of a given value. Alternatively, the term “about” means that a value can fall within a scientifically acceptable error range for that type of value, which will depend on how qualitative a measurement can be given the available tools.

All weights and weight ratios specified for oxycodone and pharmaceutically acceptable salts there of are based on the weight of a molar equivalent of oxycodone hydrochloride.

All weights and weight ratios specified for ibuprofen and pharmaceutically acceptable salts thereof are based on the weight of a molar equivalent of the free acid of ibuprofen.

The term “advertising” refers to notifying, informing, and/or apprising one or more individuals of information (e.g., the efficacy of a pharmaceutical product for treating or reducing an indication), such as by mass media, including, but not limited to, newspaper, magazine, and internet advertisements, television commercials, and billboard signs. The term “advertising” as used herein also includes including a statement that the pharmaceutical product can treat or reduce the indication in the labeling for the pharmaceutical product.

The term “marketing” refers to the act or process of selling a product, including, but not limited to, any offer for sale or sale of a product.

The term “pharmaceutical product” refers to any product containing oxyocodone or a pharmaceutically acceptable salt thereof and ibuprofen or a pharmaceutically acceptable salt thereof, including, but not limited to, pharmaceutical compositions containing the same and unit dosage forms, such as tablets and capsules, containing the same.

The term “acute pain” refers to pain that lasts or is anticipated to last a short time, typically less than a month. The term “acute pain” includes, but is not limited to, moderate, severe, and moderate to severe acute pain.

The term “acute postoperative pain” refers to acute pain resulting from surgery (such as dental surgery (e.g., molar extraction and in particular third molar extraction)). Acute postoperative pain is a physiologic reaction to tissue injury, visceral distension, or disease.

The term “patient” as used herein refers to a mammal and preferably a human.

The phrase “pharmaceutically acceptable” refers to additives or compositions that are physiologically tolerable and do not typically produce an allergic or similar untoward reaction, such as gastric upset, dizziness and the like, when administered to a mammal.

The terms “treat” and “treating” refer to reducing or relieving pain.

As used herein, the terms “effective analgesic amount” and “effective amount” refer to an amount of oxycodone or a pharmaceutically acceptable salt thereof and ibuprofen or a pharmaceutically acceptable salt thereof that, when administered to a mammal for treating pain, is sufficient to treat the pain. The “effective analgesic amount” may vary depending on the severity of pain and the mammal to be treated. Preferably, the amount of oxycodone and ibuprofen administered is effective to provide partial or complete pain relief within 30 minutes of administration. More preferably, the amount is sufficient to provide partial or complete pain relief within 22, 23, 24, 25, 26, 27, 28, or 29 minutes of administration.

Pharmaceutically acceptable salts of oxycodone include, but are not limited to, hydrochlorides, hydrobromides, hydroiodides, sulfates, bisulfates, nitrates, citrates, tartrates, bitartrates, phosphates, malates, maleates, fumarates, succinates, acetates, terephthalates, and pamoates. A preferred pharmaceutically acceptable salt of oxycodone is oxycodone hydrochloride.

The ibuprofen may be in any form, including ibuprofen USP 90% (DCI-90). Pharmaceutically acceptable salts of ibuprofen include, but are not limited to, ibuprofen salts of aluminum, calcium, potassium, and sodium.

The amount of oxycodone in the dosage forms of the present invention to be administered daily preferably ranges from about 0.025 or 0.05 to about 7.50 milligrams per kilogram of body weight (mg/kg). The amount of ibuprofen in the compositions to be administered daily preferably ranges from about 5 to about 120 milligrams per kilogram of body weight (mg/kg).

Preferably, at least 95% by weight of the oxycodone and pharmaceutically acceptable salts thereof is released from the oral dosage form after 15 minutes in FaSSIF. The maximum plasma concentration of ibuprofen is preferably reached within 1.5 hours after administration of the oral dosage form.

In a preferred embodiment, the oral dosage form contains from about 5 to about 10 mg of oxycodone or a pharmaceutically acceptable salt thereof and about 400 mg of ibuprofen or a pharmaceutically acceptable salt thereof. For example, the oral dosage form may contain about 5 or about 10 mg of oxycodone or a pharmaceutically acceptable salt thereof (e.g., oxycodone HCl) and 400 mg of ibuprofen or a pharmaceutically acceptable salt thereof. Such an oral dosage form is preferably administered to a patient 1 to 5 times daily and more preferably 1 to 4 times daily. According to one embodiment, such an oral dosage form is administered to a patient for up to 1 week.

The oral dosage forms may be tablets, pills, capsules, caplets, boluses, powders, granules, elixirs, syrups, or suspensions. The oral dosage form is preferably a solid, such as a tablet, pill, caplet, or capsule. The solid dosage forms may include pharmaceutically acceptable additives, such as excipients, carriers, diluents, stabilizers, plasticizers, binders, glidants, disintegrants, bulking agents, lubricants, plasticizers, colorants, film formers (e.g., Opadry White and Opadry II White), flavouring agents, preservatives, dosing vehicles, and any combination of any of the foregoing. Preferably, these additives are pharmaceutically acceptable additives, such as those described in Remington's, The Science and Practice of Pharmacy, (Gennaro, A. R., ed., 19th edition, 1995, Mack Pub. Co.) which is herein incorporated by reference.

When tablets containing ibuprofen and oxycodone hydrochloride were prepared, they exhibited picking defects. See, for example, Example 2A below. In particular, the logo and product identification de-bossing was picked making it difficult to read and less aesthetically pleasing. The term “picking” refers to the removal of material (such as a film fragment) from the surface of a tablet and its adherence to the surface of another object (such as another tablet or a punching machine). See pages 101 and 272 of Pharmaceutical Dosage Forms: Tablets Volume 3, edited by H. A. Lieberman and L. Lachman, Marcel Dekker, Inc. (1982). Picking may occur, for example, when tablets are compressed or tumbled. The material removed may include logos, monograms, lettering, and numbering which were intended to appear on the surface of the tablet.

It was surprisingly found that the inclusion of silicified microcrystalline cellulose in the tablet eliminated picking defects, irrespective of whether the tablets were prepared by direct compression or wet granulation methods. As a result, more expensive printing techniques are not required to prevent the picking defects. The inclusion of a mixture of microcrystalline cellulose and colloidal silicon dioxide rather than silicified microcrystalline cellulose did not, however, eliminate picking defects. It was also found that the silicified microcrystalline cellulose did not result in any loss of the direct compressibility of the formulation or slow the release of the ibuprofen or oxycodone hydrochloride upon administration.

The term “an anti-picking effective amount” refers to an amount which is sufficient to substantially eliminate picking defects. Preferably, the tablets contain an amount sufficient for them (1) to meet Acceptable Quality Limits (AQL) in accordance with ANSI/ASQC standards and/or (2) to exhibit no significant debassing or logo defects. Preferably, the number of tablets which do not meet AQL in accordance with ANSI/ASQC standards is less than 1% or 0.1% of the tablets produced.

Silicified microcrystalline cellulose acts as a filler and glidant. The term “silicified microcrystalline cellulose” refers to a particulate agglomerate of coprocessed microcrystalline cellulose and from about 0.1 to about 20% by weight of silicon dioxide, by weight of the microcrystalline cellulose. The microcrystalline cellulose and silicon dioxide in the particulate agglomerate are in intimate association with each other. The silicon dioxide portion of the silicified microcrystalline cellulose is preferably derived from silicon dioxide having an average primary particle size of from about 1 nm to about 100 cam. According to one embodiment, the average primary particle size of the silicon dioxide ranges from about 5 nm to about 40 or 50 μm. “Primary particle size” refers to the size of the particles when not agglomerated.

The silicon dioxide may have a surface area of from about 10 m²/g to about 500 m²/g, from about 50 m²/g to about 500 m²/g, or from about 175 m²/g to about 350 m²/g.

In one embodiment, the silicified microcrystalline cellulose comprises from about 0.5% to about 10% by weight of silicon dioxide, based on 100% total weight of the microcrystalline cellulose. According to another embodiment, the silicified microcrystalline cellulose comprises from about 1.25% to about 5% by weight of silicon dioxide, based on 100% total weight of the microcrystalline cellulose.

According to one embodiment, the moisture content of the silicified microcrystalline cellulose ranges from about 0.5 to about 2.5 LOD (loss on drying), from about 0.5 to about 1.8 LOD, from about 0.5 to about 1.5% LOD, or from about 0.8 to about 1.2% LOD.

Preferred silicified microcrystalline celluloses include, but are not limited to, those described in U.S. Pat. Nos. 5,725,884, 6,103,219, and 6,471,994, all of which are hereby incorporated by reference, and Prosolv SMCC 90 (which is a mixture of colloidal silicon dioxide NF and microcrystalline cellulose NF available from Penwest Pharmaceuticals Co. of Patterson, N.J.).

Suitable binders include, but are not limited to, starch, gelatin, sugars (such as sucrose, molasses and lactose), natural and synthetic gums (such as acacia, sodium alginate, carboxymethyl cellulose, methyl cellulose, polyvinylpyrrolidone, polyethylene glycol, ethylcellulose, and waxes).

Suitable glidants include, but are not limited to, talc and silicon dioxide (e.g, colloidal silicon dioxide).

Suitable disintegrants include, but are not limited to, starches, sodium starch glycolate, croscarmellose sodium, crospovidone, clays, celluloses (such as purified cellullose, methylcellulose, sodium carboxymethyl cellulose), alginates, pregelatinized corn starches, and gums (such as agar, guar, locust bean, karaya, pectin and tragacanth gums). A preferred disintegrant is sodium starch glycolate.

Suitable bulking agents include, but are not limited to, starches (such as corn starch), microcrystalline cellulose, lactose (e.g., lactose monohydrate), sucrose, dextrose, mannitol, calcium phosphate, and dicalcium phosphate.

Suitable lubricants include, but are not limited to, stearic acid, stearates (such as calcium stearate and magnesium stearate), talc, sodium fumarate, polyethylene glycol, hydrogenated cottonseed, and castor oils.

Preferred tablet formulations include those shown in the table below.

| | Concentration (percent by weight) | |
|---|---|---|
| Ingredient | Preferred | More Preferred |
| Ibuprofen | from about 64 to about | from about 70 to |
| | 77% | about 75% |
| Oxycodone Hydrochloride | from about 0.7 to about | from about 0.7 to |
| | 1.7% | about 1.7% |
| Silicified Microcrystalline | from about 15 to about | from about 15 to |
| Cellulose | 22% | about 17% |
| Sodium Starch Glycolate | from about 2.5 to about | from about 3.5 to |
| | 4.5% | about 4% |
| Stearic Acid | from about 1.5 to about | from about 2 to |
| | 3% | about 2.5% |
| Calcium Stearate | from about 0.5 to about | from about 0.6 to |
| | 1.5% | about 1% |
| Coating (e.g. Opadry ™) | from about 2 to about 5% | from about 2.5 to |
| | | about 3.5% |

Solid dosage forms may be prepared by mixing the ibuprofen and oxycodone with a pharmaceutically acceptable carrier and any other desired additives, such as by wet or dry granulation. The mixture is typically mixed until a homogeneous mixture of the oxycodone, ibuprofen, carrier, and any other desired additives is formed, i.e., until the active agents are dispersed evenly throughout the mixture. The mixture may be formed into tablets by any method known in the art (e.g., direct compression and wet granulation), including those described in Pharmaceutical Dosage Forms: Tablets, H. Liebermand and L. Lachman, 1982, which is hereby incorporated by reference.

The oral dosage forms are preferably formulated as “immediate release” dosage forms. The oral dosage forms may also be formulated as “controlled release” dosage forms. “Controlled,” “sustained,” “extended” or “time release” dosage forms are equivalent terms that describe the type of active agent delivery that occurs when the active agent is released from a delivery vehicle at an ascertainable and manipulatable rate over a period of time, which is generally on the order of minutes, hours or days, typically ranging from about sixty minutes to about 3 days, rather than being dispersed immediately upon entry into the digestive tract or upon contact with gastric fluid. A controlled release rate can vary as a function of a multiplicity of factors. Factors influencing the rate of delivery in controlled release include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract. Typical parameters for dissolution test of controlled release forms are found in U.S. Pharmacopeia standard <724>.

The following examples illustrate the invention without limitation. All parts and percentages are given by weight unless otherwise indicated.

### EXAMPLE 1

### Preparation of Oxycodone/Ibuprofen Tablets

Ibuprofen 90% (DCI-90) (454.54 mg/tablet, equivalent to 400 mg/tablet ibuprofen), oxycodone hydrochloride (5.17 mg/tablet, equivalent to 5.00 mg/tablet oxycodone hydrochloride), and povidone USP (available as Plasdone K-30 from International Specialty Products Corporation of Wayne, N.J.) (4.55 mg/tablet) were mixed for 5 minutes. The ingredients were granulated with purified water. After drying the wet granules, colloidal silicon dioxide NF (2.30 mg/tablet), microcrystalline cellulose NF (199.84 mg/tablet), and stearic acid NF (13.60 mg/tablet) were added. The blend was compressed and the tablets were coated with an aqueous coating concentrate (Colorcon Formulation No. YSI-7085 or YSI-741 1, Colorcon of West Point, Pa.) (27.00 mg/tablet).

### EXAMPLE 2

Ibuprofen USP 90% (DCI-90) (444.40 mg/tablet, equivalent to 400 mg/tablet ibuprofen), oxycodone hydrochloride USP (5.10 mg/tablet), and povidone USP (4.50 mg/tablet) were mixed in a high shear granulator. The ingredients were granulated with purified water and the wet mass dried using a fluid bed drier. The dried granules were milled and mixed in a twin shell blender with colloidal silicon dioxide NF (2.80 mg/tablet), sodium starch glycolate NF (22.80 mg/tablet), microcrystalline cellulose NF (40.90 mg/tablet), lactose monohydrate NF (41.40 mg/tablet), stearic acid NF (13.60 mg/tablet), and a portion of calcium stearate NF (7.50 mg/tablet) for 35 minutes. The remaining portion of calcium stearate NF was added to the blender and mixed for an additional 5 minutes. The blend was compressed using a rotary tablet press. The tablets were then coated with Opadry White (17.50 mg/tablet) with a perforated coating pan.

### EXAMPLE 2A

Tablets were prepared according to the procedure in Example 2 without the Opadry White coating. Once all of the materials were added together, they were blended in a 10-ft³ blender rotating at 20 rpm for 40 minutes. The blend was then compressed with a rotary tablet press. Sticking was observed almost immediately during the compression operation. After 10 minutes, tablet appearance was deemed unacceptable and the compression was discontinued.

### EXAMPLE 3

Ibuprofen USP 90% (DCI-90) (222.22 mg/tablet, equivalent to 200 mg/tablet ibuprofen), oxycodone hydrochloride USP (5.10 mg/tablet), and povidone USP (2.25 mg/tablet) were mixed in a high shear granulator. The ingredients were granulated with purified water and the wet mass dried using a fluid bed drier. The dried granules were milled and mixed in a twin shell blender with colloidal silicon dioxide NF (1.40 mg/tablet), sodium starch glycolate NF (11.40 mg/tablet), microcrystalline cellulose NF (28.45 mg/tablet), lactose monohydrate NF (28.63 mg/tablet), stearic acid NF (6.80 mg/tablet), and a portion of the calcium stearate NF lot (3.75 mg/tablet) for 35 minutes. The remaining portion of calcium stearate was added to the blender and mixed for an additional 5 minutes. The blend was compressed by a rotary tablet press. The tablets were then coated with Opadry White (9.30 mg/tablet) with a perforated coating pan.

### EXAMPLE 4

Ibuprofen USP 90% (DCI-90) (444.40 mg/tablet, equivalent to 400 mg/tablet ibuprofen), oxycodone hydrochloride USP (5.10 mg/tablet), and povidone USP (4.50 mg/tablet) were mixed in a high shear granulator. The ingredients were granulated with purified water and the wet mass dried using a fluid bed drier. The dried granules were milled and mixed in a twin shell blender with colloidal silicon dioxide NF (2.80 mg/tablet), sodium starch glycolate NF (22.80 mg/tablet), microcrystalline cellulose NF (40.90 mg/tablet), lactose monohydrate NF (41.00 mg/tablet), stearic acid NF (13.60 mg/tablet), and a portion of the calcium stearate NF lot (7.50 mg/tablet) for 35 minutes. The remaining portion of calcium stearate was added to the blender and mixed for an additional 5 minutes. The blend was compressed by a rotary tablet press. The tablets were then coated with Opadry II White (17.50 mg/tablet) with a perforated coating pan.

### EXAMPLE 4A

The procedure of Example 4 was repeated with 10.2 mg/tablet of oxycodone hydrochloride USP, 22.8 mg/tablet of sodium starch glycolate NF, and 35.8 mg/tablet microcrystalline cellulose NF.

### EXAMPLE 5

Prosolv SMCC 90 (which is a mixture of colloidal silicon dioxide NF and microcrystalline cellulose NF available from Penwest Pharmaceuticals Co. of Patterson, N.J.) (104.2 mg/tablet) and oxycodone hydrochloride USP (5.0 mg/tablet) were mixed in a twin shell blender for 10 minutes. A portion (approximately 25% or 112.5 mg/tablet) of ibuprofen USP 90% (DCI-90) (total 450.0 mg/tablet) was added and mixed for 10 minutes. Stearic acid NF (13.6 mg/tablet), calcium stearate NF (4.5 mg/tablet), sodium starch glycolate NF (22.7 mg/tablet), and the remaining ibuprofen USP 90% (approximately 337.5 mg/tablet) were added to the blender and mixed for 40 minutes. The blend was compressed by a rotary tablet press. The tablets were then coated with Opadry II White (18.0 mg/tablet) with a perforated coating pan.

### EXAMPLE 6

The procedure of Example 5 was repeated with 10.0 mg/tablet of oxycodone hydrochloride USP and 99.2 mg/tablet of Prosolv SMCC 90.

### EXAMPLE 7

The following two clinical studies were performed to evaluate the analgesic efficacy of a unitary formulation containing oxycodone HCl and ibuprofen.

Study 1

498 patients were randomized in a double-blind, placebo- and active-controlled, multicenter, parallel study. Patients with moderate to severe pain following surgical removal of at least 2 ipsilateral bony impacted third molars received a single dose of oxycodone HCl/ibuprofen 5/400 mg combination (as a single tablet) (prepared as described in Example 4), 5 mg oxycodone HCl, 400 mg ibuprofen, or placebo. The primary efficacy paramaters of total pain relief and sum of pain intensity difference were evaluated for 6 hours postdose.

The 5 mg oxycodone HCl/400 mg ibuprofen tablet (21.4 minutes) resulted in an earlier onset of analgesia compared with 400 mg ibuprofen (29.7 minutes) (P<0.01) or 5 mg oxycodone HCl (>360 minutes) (P<0.001). The oxycodone HCl/ibuprofen tablet had a 28% faster median time to onset of pain relief than did ibuprofen alone (21.4 v. 29.7 minutes).

Study 2

In a multi-site, double-blind, parallel-group study, patients with moderate to severe pain following surgical removal of at least 2 ipsilateral bone impacted third molars were randomized to a single dose of oxycodone HCl/ibuprofen 5/400 mg (single tablet) (n=171) (prepared as described in Example 4), oxycodone HCl/ibuprofen 10/400 mg (single tablet) (prepared as described in Example 4A) (n=169), 400 mg ibuprofen (n=171), 5 mg oxycodone HCl (n=57), 10 mg oxycodone HCl (n=57), and placebo (n=57) and evaluated for 6 hours postdose. The median times to onset of pain relief for 5 mg oxycodone HCl/400 mg ibuprofen, 10 mg oxycodone HCl/400 mg ibuprofen, 400 mg ibuprofen, 5 mg oxycodone HCl, and 10 mg oxycodone HCl were 25.4, 22.5, 28.0, 67.3, and 63.4 minutes, respectively.

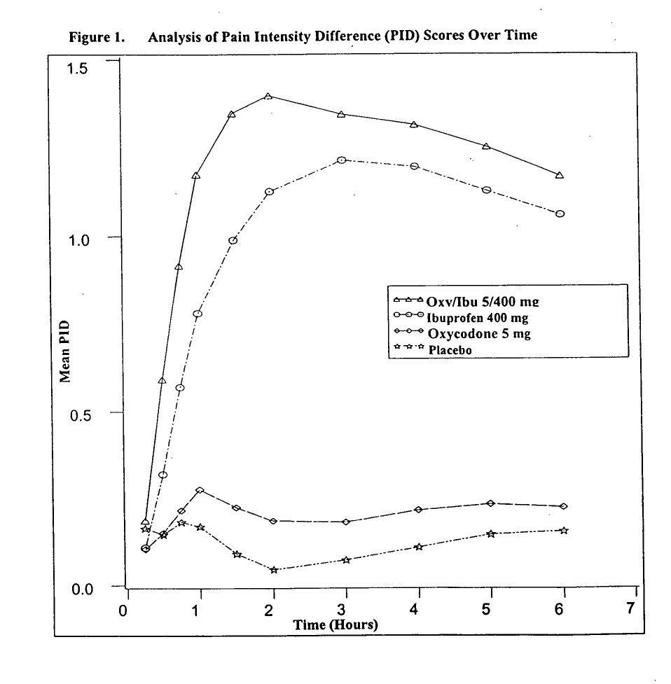

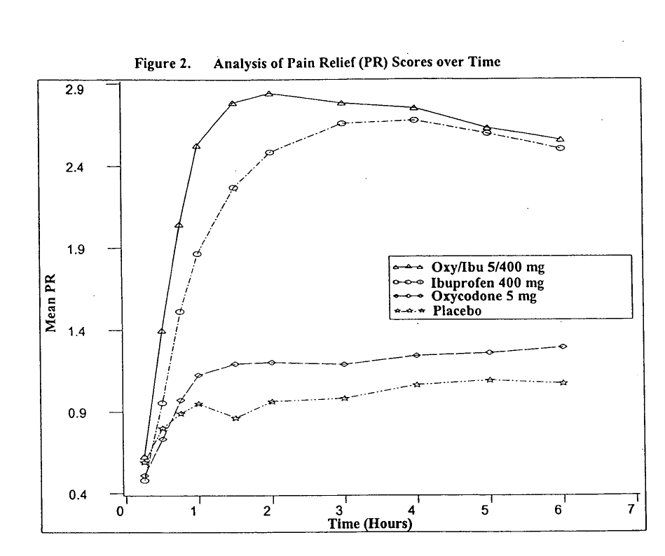

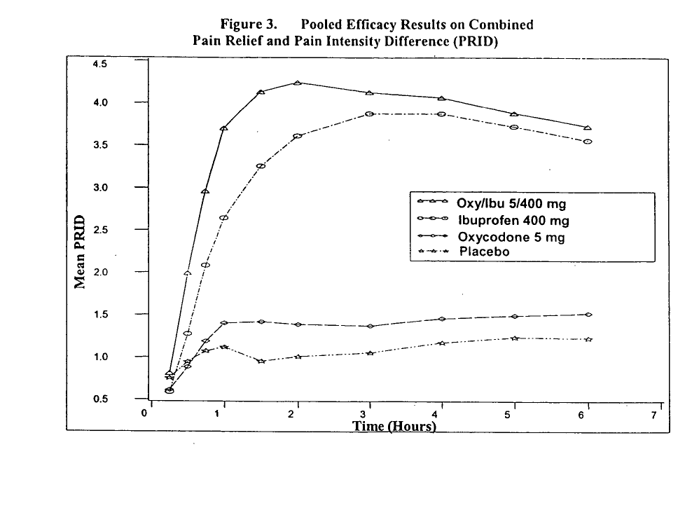

The results from these two studies were pooled. FIGS. 1-3 show the pain intensity difference (PID), pain relief (PR) scores, and combined pain relief and pain intensity difference (PRID), respectively, over 6 hours for the pooled data for 5 mg oxycodone/400 mg ibuprofen, 400 mg ibuprofen, 5 mg oxycodone HCl, and placebo. In the pooled analysis, the median time to onset of pain relief for 5 mg oxycodone HCl/400 mg ibuprofen was 22.9 minutes, which was significantly (p<0.05) shorter than for ibuprofen alone (29.0 minutes). The median time could not be estimated for the oxycodone and placebo groups as fewer than 50% of the patients in these groups experienced pain relief.

### EXAMPLE 8

A randomized, two-way crossover study in healthy male subjects was performed. Subjects received the following treatments in random order:

A. one tablet prepared by the procedure in Example 1 (5 mg oxycodone HCl and 400 mg ibuprofen) with 240 mL of water after overnight fast, and

B. one oxycodone tablet (5 mg) and 2×200 mg immediate release Medipren® ibuprofen caplets (available from Johnson & Johnson of New Brunswick, N.J.) with 240 mL of water after overnight fast.

There was a 7-day washout between periods.

24 male subjects were entered into the study. All the subjects completed the study. The average age of the subjects was 25±5 years (range, 20-38 years).

Blood samples were taken at 0.0 hour (pre-dose) and 0.5, 1, 1.5, 2, 3, 4, 6, 7, and 10 hours after the administration of the two treatments. Blood samples were collected and plasma was analyzed for oxycodone and total ibuprofen concentrations.

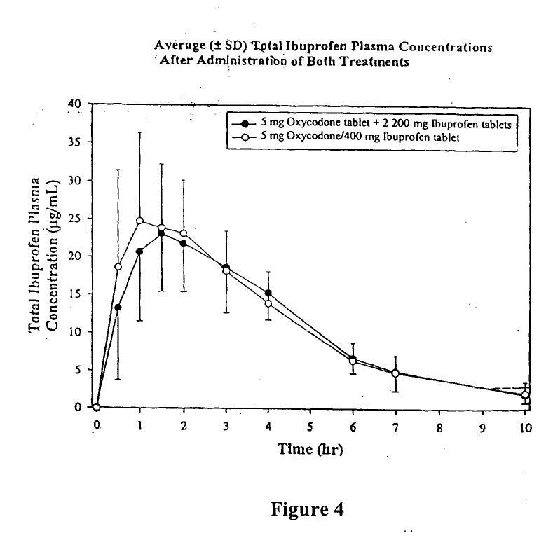

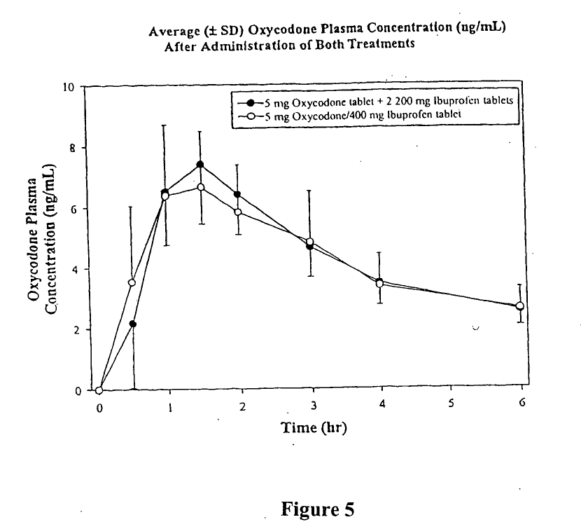

The average plasma concentration time profiles for ibuprofen and oxycodone are shown in FIGS. 4 and 5, respectively. The average Cₘₐₓ, AUC₀₋ₜ, AUC_{0-∞}, Tₘₐₓ, and T_{1/2} (±standard deviation) for oxycodone and ibuprofen, based on the two one-sided test procedure using log-transformed data, are shown in Tables 1 and 2, respectively.

TABLE 1
Ibuprofen Profile

| | | 5 mg Oxycodone Tablet |
|---|---|---|
| | Tablet | with 2 × 200 mg Ibuprofen |
| | of Example 1 | (Medipren ®) Tablets |
| Cₘₐₓ (μg/mL) | 30.6 ± 8.8 | 28.1 ± 7.5 |
| | (90% C.I.*: 97-121) | |
| AUC₀₋ₜ (μg · hr/mL) | 112.4 ± 22.2 | 109.5 ± 16.7 |
| | (90% C.I*: 96-108) | |
| AUC_{0-∞} (μg · hr/mL) | 122.4 ± 28..5 | 115.8 ± 19.8 |
| | (90% C.I.*: 97-113) | |
| Tₘₐₓ (hr) | 1.4 ± 0.9 | 2.2 ± 1.5 |
| T_{1/2} (hr) | 2.1 ± 0.6 | 1.9 ± 0.4 |

*—C.I. = “Confidence Interval”

TABLE 2
Oxycodone Profile

| | | 5 mg Oxycodone Tablet |
|---|---|---|
| | Tablet | with 2 × 200 mg Ibuprofen |
| | of Example 1 | (Medipren ®) Tablets |
| Cₘₐₓ (ng/mL) | 7.5 ± 1.8 | 8.0 ± 1.7 |
| | (90% C.I.: 85-102) | |
| AUC₀₋ₜ (ng · hr/mL) | 19.4 ± 5.1 | 19.2 ± 3.6 |
| | (90% C.I.: 91-110) | |
| AUC_{0-∞} (ng · hr/mL) | 36.5 ± 10.7 | 36.4 ± 5.2 |
| | (90% C.I.: 90-111) | |
| Tₘₐₓ (hr) | 1.4 ± 0.6 | 1.4 ± 0.4 |
| T_{1/2} (hr) | 2.8 ± 0.8 | 2.8 ± 0.9 |

### EXAMPLE 9

The objective of this study was to investigate the effects of potential drug-drug interaction between ibuprofen and oxycodone on their permeability characteristics across Caco-2 cell monolayers. Ibuprofen/oxycodone HCl tablets containing 5 mg of oxycodone (hydrochloride salt, all mass concentrations of oxycodone used in this study were based on the total weight of the hydrochloride salt, not on its free base) and 400 mg of ibuprofen were used. The dose ratio of oxycodone to ibuprofen was 1:80 (w/w). The molecular weight of oxycodone hydrochloride is 351.87 and the molecular weight of ibuprofen is 206.28; therefore, the molar ratio of oxycodone/ibuprofen (5 mg/400 mg) is 1:136. According to the literature, the absolute bioavailability of oxycodone was reported to be 87%, and the bioavailability of ibuprofen was reported to approach 100%. Leow, K. P., Smith, M. T., Williams, B. and Cramond, T., “Single-Dose and Steady State Pharmacokinetics and Pharmacodynamics of Oxycodone in Patients with Cancer”, Clin. Pharmacol. Ther., 52: 487-495 (1992); Hall, S. D., Rudy, A. C., Knight, P. M. and Brater, D. C., “Lack of Presysternic Inversion of (R)- to (S)-Ibuprofen in Humans”, Clin. Pharmacol. Therap., 53: 393-400 (1993). Caco-2 cell monolayers have been used as a model of intestinal mucosa for predicting oral drug absorption (P. Artursson. Epithelial transport of drugs in cell culture. I: A model for studying the passive diffusion of drugs over intestinal absorptive (Caco-2) cells. J Pharm Sci. 79:476-482. (1990)). The transport experiments of oxycodone and ibuprofen were conducted in the apical (AP) to basolateral (BL) direction across Caco-2 cell monolayers.

Materials

The Caco-2 cell monolayers were grown in the laboratory. Hank's balanced salt solution buffer (HBSS) was prepared in the laboratories.

Preparation of Dosing Solutions of Oxycodone and Ibuprofen

Solutions containing 0.02 mg/ml oxycodone hydrochloride and 0, 0.8, 1.6, or 3.2 mg/ml ibuprofen were prepared as follows. One stock solution of oxycodone in DMSO (10 mg/ml, hydrochloride salt) was prepared. Two stock solutions of ibuprofen in DMSO (100 mg/ml and 200 mg/ml) were prepared. The solutions of oxycodone (0.02 mg/ml, hydrochloride salt) were made by diluting the stock solutions in HBSS (pH=6.8). A total of 40 and 80 μl of ibuprofen DMSO stock solutions (100 mg/ml) and 80 μl of ibuprofen DMSO stock solution (200 mg/ml ibuprofen) were transferred to 5 ml of solutions of oxycodone (0.02 mg/ml), respectively. The concentrations of ibuprofen in dosing solutions were 0, 0.8, 1.6 and 3.2 mg/ml, respectively. The concentration of DMSO in all the donor and receiver solutions was adjusted to 1.6%.

The solutions of ibuprofen (0.2 mg/ml) were made transferring 200 μl the ibuprofen stock solution (10 mg/ml) into 10 ml of HBSS (pH=6.8). 0, 2.5, 5, and 10 μl of the oxycodone DMSO stock solution (10 mg/ml) were transferred to 10 ml of the aforementioned solutions of ibuprofen (200 μl/ml), respectively. The concentrations of oxycodone (hydrochloride salt) in these solutions were 0, 2.5, 5, and 10 μl/ml, respectively, and the concentration of DMSO in the donor compartment was about 2%. The concentration of DMSO in the receiver solution was adjusted to 2%.

Experiment

The transport experiments were performed using Caco-2 cell monolayers grown on a 12-well TRANSWELL® system (Costar, Cambridge, Mass.). All experiments were done at 37° C. with constant mixing in a water shaker-bath (60 rpm). Both the AP and the BL compartments of each insert were washed twice with 37° C. HBSS (pH=7.4) and incubated for 15 minutes. The pH value of HBSS was 6.8 for the donor (AP) and 7.4 for the receiver (BL) solutions. 500 μl of solution was added to the AP compartment and 1500 μl of solution was placed in the BL compartment. Aliquots (750 μl) were withdrawn from the receiver side at 20-minute time intervals to 80 minutes. HBSS was replaced in the receiver side after sampling. Aliquots (50 μl) were withdrawn from the donor side at 10 minutes and 80 minutes. Each treatment was performed in triplicate. The membrane integrity of the cell monolayers was monitored before and after the transport experiments by measuring the transepithelial electric resistant (TEER) of the cell monolayers. Samples then underwent LC/MS/MS analysis.

The transport of oxycodone (0.02 mg/ml) across Caco-2 cell monolayers in the AP-to-BL direction was measured in the absence and presence of increasing concentrations of ibuprofen (0, 0.8 mg/ml, 1.6 mg/ml, and 3.2 mg/ml). The dose ratios of oxycodone to ibuprofen were 0, 1:40, 1:80, and 1:160 (w/w), respectively.

The transport of ibuprofen (0.2 mg/ml) across Caco-2 cell monolayers in the AP-to-BL direction was conducted in the absence and presence of increasing concentrations of oxycodone (0, 2.5 μg/ml, 5 μg/ml, and 10 μg/ml). The dose ratios of oxycodone to ibuprofen were 0, 1:80, 1:40, and 1:20 (w/w), respectively.

Apparent permeability coefficient (Papp) values were calculated using the equation:
Pₐₚₚ=ΔQ/Δt/(A*C₀) (1)
where ΔQ/Δt is the linear appearance rate of mass in the receiver solution, A is the filter/cell surface area (1 cm²), and C₀ is the initial concentration of the test compounds.

Statistical analyses were performed using Student's two-tailed t-test between two mean values. A probability of less than 0.05 (p<0.05) was considered to be statistically significant.

Results

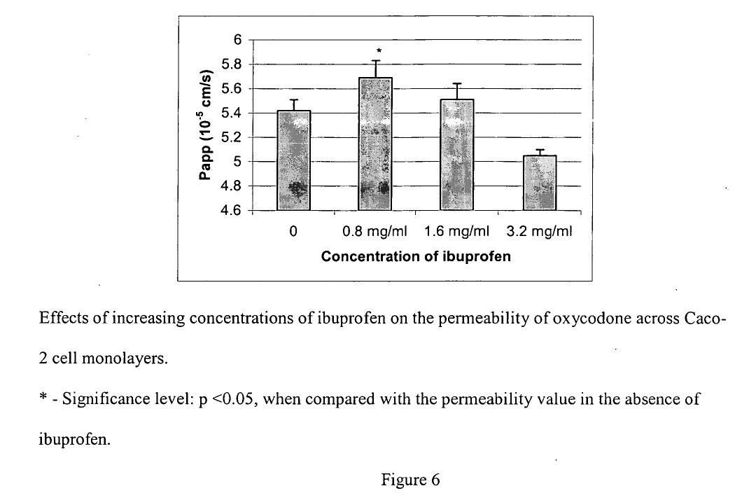

As shown in Table 3 below and FIG. 6, oxycodone had a Pₐₚₚ value of 5.42±0.09×10⁻⁵ cm/s across Caco-2 cell monolayers. In the presence of 0.8 mg/ml of ibuprofen, the permeability of oxycodone was enhanced to 5.69±0.14×10⁻⁵ cm/s. Ibuprofen at the concentration of 1.6 mg/ml appeared to marginally increase the permeability of oxycodone although the effects were not significant. When 3.2 mg/ml of ibuprofen was prepared in HBSS, ibuprofen formed a precipitate and slightly decreased the permeability of oxycodone to 5.05±0.05×10⁻⁵ cm/s. A portion of oxycodone might be coprecipitated from the transport media and result in less amount of oxycodone available for transport, thus decreasing the overall permeability of oxycodone. The membrane integrity of Caco-2 cell monolayers was monitored before and after the transport experiments. The TEER values of cell monolayers were in the range of 980-1002 Ωcm² before the transport experiments and the values were not changed after the transport experiments were conducted. Therefore, ibuprofen and oxycodone at the concentrations used in the experiment did not compromise the integrity of Caco-2 cell mono layers.

TABLE 3
Permeability of Oxycodone across Caco-2 Cell
Monolayers in the Absence and Presence of
Increasing Concentrations of Ibuprofen

| | Apparent permeability coefficients |
|---|---|
| Concentration of ibuprofen in the | of oxycodone (10⁻⁵ cm/s) |
| transport medium (mg/ml) | (±standard deviation) (n = 3) |
| 0 | 5.42 ± 0.09 |
| 0.8 | 5.69 ± 0.14 |
| 1.6 | 5.51 ± 0.13 |
| 3.2 | 5.05 ± 0.05 |

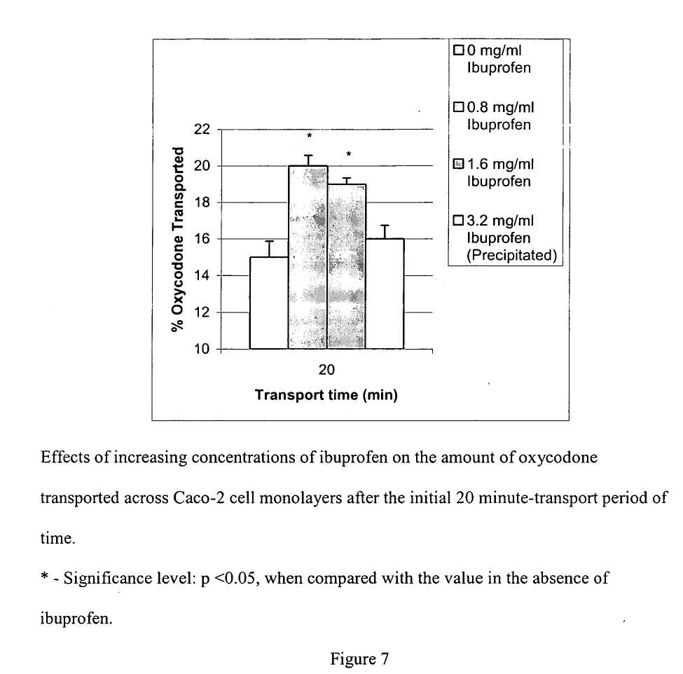

Although ibuprofen only exhibited a marginal effect on the overall permeability of oxycodone over the 80-minute transport period of time, it significantly enhanced the initial transport rate of oxycodone across Caco-2 cell monolayers. As shown in Table 4 and FIG. 7, after the initial 20-minute transport period of time, the percentage of transported oxycodone from apical to basolateral compartment was increased from 15% to 20% and 19% in the presence of 0.8 mg/ml and 1.6 mg/ml of ibuprofen, respectively. Ibuprofen at the concentration of 3.2 mg/ml did not increase the transport of oxycodone due to its precipitating from the transport media. Since the rate of onset of action of a drug is dependent on the time for the drug to be absorbed and accumulated to its low concentration limit of the therapeutics window, the initial absorption rate of oxycodone and ibuprofen in the GI tract might play an important role in its faster onset of action. The increased initial transport rate of oxycodone by ibuprofen may contribute to the fast onset of action of oxycodone/ibuprofen formulation.

TABLE 4
Permeability of Oxycodone across Caco-2 Cell
Monolayers in the Absence and Presence of
Increasing Concentrations of Ibuprofen after 20 minutes

| | Apparent permeability coefficients |
|---|---|
| Concentration of ibuprofen in the | of oxycodone (10⁻⁵ cm/s) |
| transport medium (mg/ml) | (±standard deviation) (n = 3) |
| 0 | 1.5 ± 0.09 |
| 0.8 | 2.0 ± 0.06 |
| 1.6 | 1.9 ± 0.03 |
| 3.2 | 1.6 ± 0.07 |

Oxycodone is a tertiary amine molecule. Its pKa is about 9. It is highly charged at all physiological pH. At the oxycodone/ibuprofen dose ratios of 1:40 (oxycodone: 0.02 mg/ml, ibuprofen 0.8 mg/ml) and 1:80 (oxycodone: 0.02 mg/ml, ibuprofen 1.6 mg/ml), the molar ratios of oxycodone to ibuprofen in the transport buffer were 1:68 and 1:136, respectively. Each oxycodone molecule in solution had a large number of ibuprofen molecules surrounding it. Oxycodone may interact with ibuprofen, a benzeneacetic acid derivative, to form a less polar organic ion pair, thus increasing its biomembrane permeation rates.

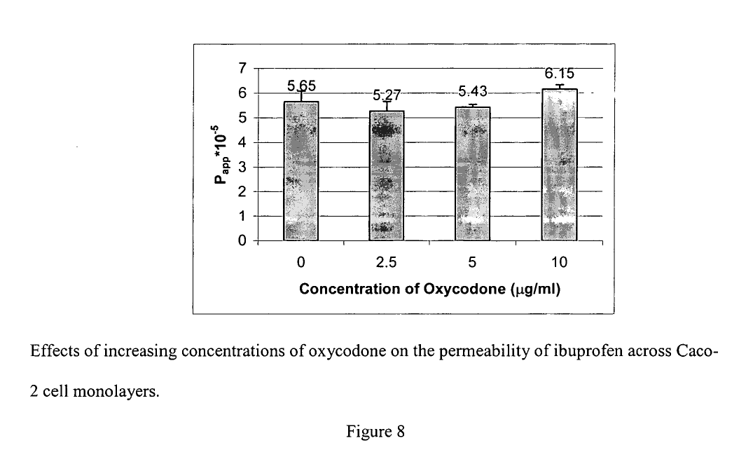

Ibuprofen has been reported to be a highly permeable drug (FDA CDER, Guidance for Industry: Waiver of In Vivo Bioavailability and Bioequivalence Studies for Immediate Release Solid Oral Dosage Forms Containing Certain Active Moieties/Active Ingredients Based on a Biopharmaceutics Classification System. Food and Drug Administration: Rockville, Md., 2000. 1197-1204). As noted above, the bioavailability of ibuprofen approaches 100%. As shown in Table 5 below and FIG. 8, ibuprofen had a Caco-2 permeability value of 5.65±0.43×10⁻⁵ cm/s, which is consistent with its highly permeable characteristics. In the presence of oxycodone at oxycodone/ibuprofen dose ratios of 1:80, 1:40, and 1:20 (w/w), the Caco-2 permeability of ibuprofen was no different from the control (Table 5 and FIG. 8). At the oxycodone/ibuprofen dose ratios of 1:80, 1:40, and 1:20 (w/w) in the transport buffer, the molar ratios of oxycodone to ibuprofen were 1:136, 1:68, and 1:34, respectively.

TABLE 5
Permeability of Iburpofen across Caco-2 Cell
Monolayers in the Absence and Presence of
Increasing Concentrations of Oxycodone

| | Apparent permeability coefficients of |
|---|---|
| Concentration of oxycodone in | ibuprofen (10⁻⁵ cm/s) |
| the transport medium (μg/ml) | (±standard deviation) (n = 3) |
| 0 | 5.65 ± 0.43 |
| 2.5 | 5.27 ± 0.39 |
| 5 | 5.43 ± 0.11 |
| 10 | 6.15 ± 0.18 |

In conclusion, ibuprofen increased the initial transport rates of oxycodone across Caco-2 cell monolayers. The fast accumulation of oxycodone in patients may result in a relief.

### EXAMPLE 10

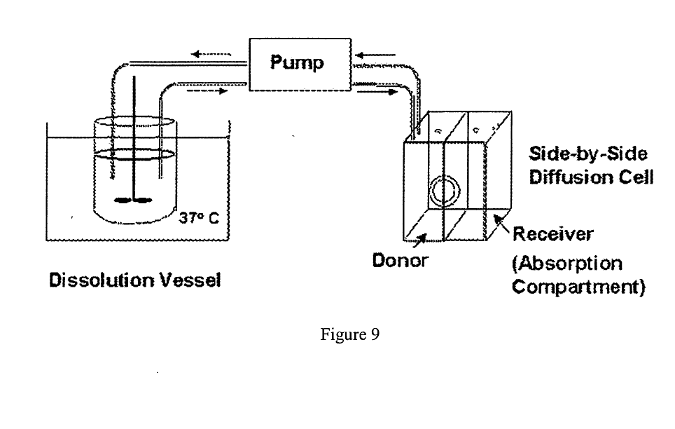

The dissolution and Caco-2 cell monolayer permeation characteristics of ibuprofen and oxycodone from unitary tablets containing 400 mg ibuprofen and 5 mg of oxycodone hydrochloride as prepared in Example 4 (hereafter referred to as the “5/400 unitary tablets”), tablets containing 200 mg of ibuprofen (Nuprin® tablets), and tablets containing 5 mg oxycodone hydrochloride (Roxicodone™ tablets) were compared in the continuous dissolution/Caco-2 cell monolayer system shown in FIG. 9. The continuous dissolution/Caco-2 system includes a Vankel dissolution apparatus (I or II) (available from Varian, Inc. of Cary, N.C.) and a side-by-side diffusion cell. In this system, dissolution and permeation of a drug across Caco-2 cell monolayers occurs continuously. Therefore, monitoring of accumulative of drug appearing in the receiver side of Caco-2 cell monolayers may be predictive of oral drug absorption of a dosage form.

Experimental

Caco-2 cell monolayers were grown in the laboratory. Fasted state simulated small intestinal fluid (FaSSIF) buffer and Hank's balanced salt solution buffer (HBSS) were prepared in the laboratory as described in J. B. Dressman, G. L. Amidon, C. Reppas and V. P. Shah, “Dissolution testing as a prognostic tool for oral drug absorption: immediate release dosage forms”, Pharm Res. 15:11-22 (1998); and F. Tang and R. T. Borchardt, “Characterization of the efflux transporter(s) responsible for restricting intestinal mucosa permeation of a coumarinic acid-based cyclic prodrug of the opioid peptide DADLE”, Pharm. Res. 19:787-793 (2002).

FaSSIF buffer has been used as the bio-relevant buffer to predict the in vivo performance of an orally administered dosage form (J. B. Dressman, G. L. Amidon, C. Reppas and V. P. Shah, “Dissolution testing as a prognostic tool for oral drug absorption: immediate release dosage forms”, Pharm Res. 15:11-22 (1998)). FaSSIF buffer was also found to be compatible with Caco-2 cell monolayers (F. Ingels, S. Deferme, E. Destexhe, M. Oth, G. Van den Mooter and P. Augustijns. Simulated intestinal fluid as transport medium in the Caco-2 cell culture model. Int J Pharm. 232:183-192 (2002)). Therefore, the dissolution studies were conducted in FaSSIF buffer in a USP apparatus II (50 rpm, 37° C.). As shown in FIG. 9, in each dissolution vessel, one 5/400 unitary tablet, two Nuprin® tablets (200 mg ibuprofen per tablet, available from Bristol-Myers Squibb Co. of New York, N.Y.), one Roxicodone™ tablet (available from Roxane Laboratories, Inc. of Columbus, Ohio) , or the combination of two Nuprin® tablets and one Roxicodone™ tablet was dissolved in 500 ml of FaSSIF buffer in USP apparatus I (100 rpm) at 37° C., respectively. The dissolution medium was filtered through a 10 μm dissolution filter and transferred via a peristaltic pump to the donor compartment of the side-by-side diffusion cell. Mounted between the donor and receiver compartments of the diffusion cell was a Caco-2 cell monolayer, which was grown onto a polycarbonate Snapwell® filter (available from Costar of Cambridge, Mass.) and cultured for 21-28 days. During the dissolution-permeation study, the dissolution medium was continuously recirculated from the donor compartment back to the dissolution vessel, therefore, the drug concentration in the donor compartment of the side-by-side diffusion cell was simultaneously changing as that in the dissolution buffer. The volume of media in the donor compartment of the side-by-side diffusion cell was maintained at 7 ml. The receiver compartment of the side-by-side diffusion cell was filled with 7 ml of HBSS. Aliquots (5 ml) were taken from the dissolution media at 5, 10, 15, 20, 30, 40, 50, and 60 minutes. 4 ml of HBSS were taken from the receiver side of the diffusion cell at 8, 13, 18, 23, 33, 43, 53, and 63 minutes taking into consideration that it took about 3 minutes to circulate drug from the dissolution vessel to the Caco-2 cell monolayer surface. 4 ml pre-warmed 37° C.-HBSS was replaced back to the receiver compartment. Samples were analyzed using HPLC or LC/MS. The low limit of quantification (LLQ) was 5 ng/ml for oxycodone LC/MS analysis. Drug concentrations below LLQ were considered as 0 ng/ml in the calculations.

Mathematical Model

In a sink condition, the drug concentration in dissolution buffer can be calculated using simplified Noyes-Whitney equation 2
dC/dt=K×Cs (2)
where K is the apparent dissolution rate constant for a formulation and Cs is the solubility of the drug substance in the dissolution buffer.

Therefore, the concentration of drug at time t (Ct) can be calculated according to equation 3.
Ct=K×Cs×t (3)

Drug permeability across the Caco-2 monolayer is calculated using modified Fick's First Law, equation 4
dM/dt=Papp×A×Ct (4)
where dM/dt is the rate of amount drug appearing in the receiver side, Papp is the apparent drug permeability constant across Caco-2 cell monolayers, A is the surface area of Caco-2 cell monolayer, which is 1 cm² for Snapwell® system, and Ct is the drug concentration in the donor compartment, which is equal to the concentration in the dissolution buffer, and is calculated in equation 2.

Equation 3 is substituted into equation 4 to yield,
dM/dt=Papp×A×K×Cs×t (5)

Integration of equation 5 yields
Mt=½×Papp×A×K×Cs×t² (6)
where Mt is the accumulative amount of drug in the receiver side of the side-by-side diffusion cell. Mt integrates the contributions of dissolution and permeation processes into overall drug absorption kinetics. Therefore, monitoring of Mt may be predictive of oral drug absorption of a dosage form.

Statistical analyses were performed using Student's two-tailed t-test between two mean values. A probability of less than 0.05 (p<0.05) was considered to be statistically significant.

Results

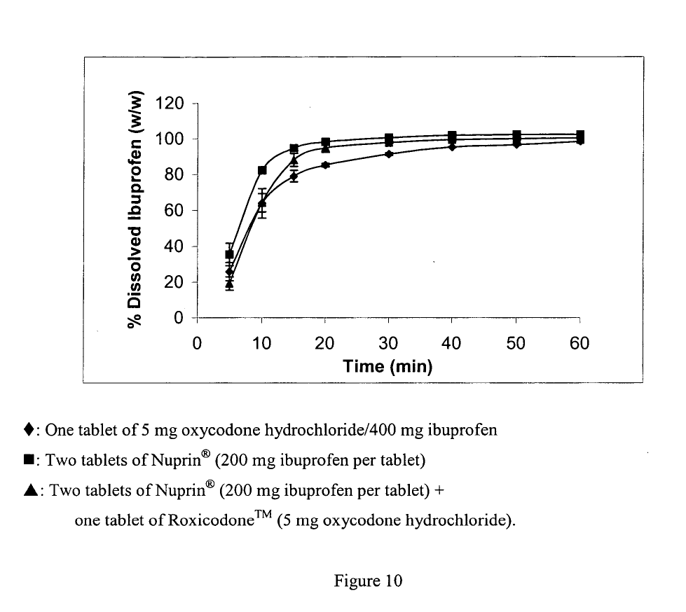

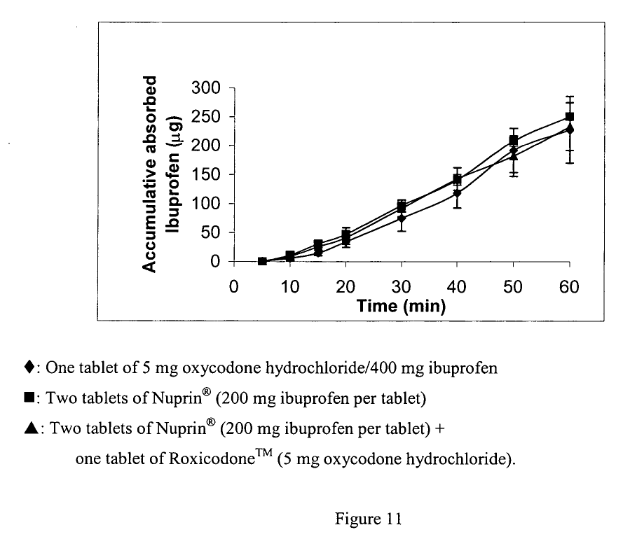

FIG. 10 shows the dissolution rates of ibuprofen from the 5/400 unitary tablets, Nuprin® tablets, and the combination of Nuprin® and Roxicodone™ tablets. All formulations had rapid ibuprofen dissolution rates in the FaSSIF buffer, i.e., more than 80% of ibuprofen was dissolved in 20 minutes. The dissolved ibuprofen into dissolution buffer from all formulations approached 100% at the later time points of 40, 50, and 60 minutes. The absorption data (FIG. 11) for ibuprofen in the dissolution/Caco-2 cell monolayer system were consistent with the dissolution results. As shown in FIG. 11, the accumulative amounts of absorbed ibuprofen in the receiver side of the Caco-2 diffusion system were similar among the three treatments.

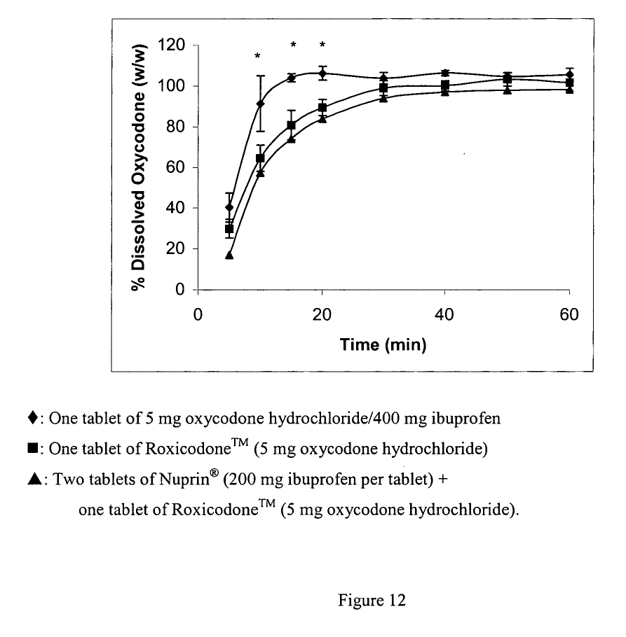

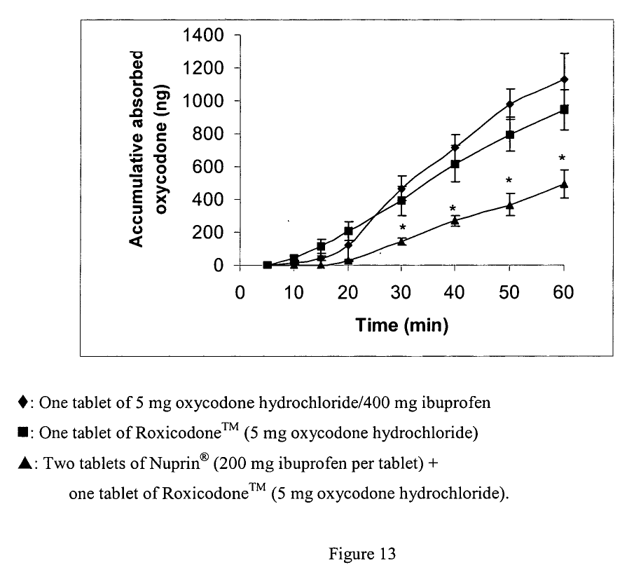

Dissolution rates of oxycodone from the 5/400 unitary tablets, the Roxicodone™ tablets, and the combination of Nuprin® and Roxicodone™ tablets were rapid. As shown in FIG. 12, more than 90% of the oxycodone was dissolved within 30 minutes for all three treatments. The dissolution rates of oxycodone from the 5/400 unitary tablets were extremely fast, i.e., 100% of oxycodone was dissolved in 15 minutes. The amounts of oxycodone dissolved from the 5/400 unitary tablets were greater than the amounts of oxycodone from Roxicodone™ tablets and the combination of Nuprin® and Roxicodone™ tablets at 10, 15, and 20 minutes (FIG. 12). FIG. 13 shows the accumulative amount of oxycodone in the receiver side of the Caco-2 system. The accumulative amounts of absorbed oxycodone from the 5/400 unitary tablets exhibited a trend of greater accumulation than from the other two treatments (FIG. 13). The accumulative amount of oxycodone appearing in the receiver compartment of Caco-2 system for the treatment of the combination of Nuprin® and Roxicodone™ was less than the accumulative amounts of oxycodone for the 5/400 unitary tablets and Roxicodone™ treatments at the time points of 30, 40, 50, and 60 minutes (FIG. 13). As discussed in the Mathematical Model section, the accumulative amount (Mt) of drug in the receiver side of dissolution/Caco-2 cell monolayer system is predictive of the oral drug absorption of a dosage form. Therefore, the aforementioned data may be indicative of the faster oral absorption of oxycodone from the 5/400 unitary tablets than the combination of Nuprin® and Roxicodone™ tablets. Since oxycodone was included in the 5/400 unitary tablets formulation to improve the anti-pain effects of ibuprofen, the faster absorption rate of oxycodone may result in the faster onset of action of 5/400 unitary tablets than the combination of Nuprin® and Roxicodone™.

The faster dissolution rate and greater amount of absorbed oxycodone from the 5/400 unitary tablets in the dissolution/Caco-2 cell monolayer system suggests rapid oral absorption of oxycodone from the 5/400 unitary tablets might be the potential reason for the fast onset of action of this drug formulation.

All references cited herein are incorporated by reference. To the extent that a conflict may exist between the specification and the reference the language of the disclosure made herein controls.

1. A method of treating a patient suffering from acute pain comprising the steps of:
(a) marketing to doctors an ibuprofen/oxycodone unitary oral dosage form comprising about 5 mg oxycodone hydrochloride and about 400 mg ibuprofen for the treatment of acute pain and as having a faster onset of pain relief than contemporaneous administration of a first oral dosage form comprising about 5 mg oxycodone hydrochloride and a second oral dosage form comprising about 400 mg ibuprofen;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient.

2. The method of claim 1, wherein step (a) further comprises marketing the ibuprofen/oxycodone unitary oral dosage form as providing partial or complete pain relief within 25 minutes after administration.

3. The method of claim 1, wherein step (a) comprises marketing the ibuprofen/oxycodone unitary oral dosage form for the treatment of moderate, severe, or moderate to severe acute pain.

4. The method of claim 1, wherein step (a) comprises marketing the ibuprofen/oxycodone unitary oral dosage form for the treatment of acute postoperative pain.

5. A method for treating a patient suffering from acute pain comprising the steps of:
(a) providing to the marketplace an ibuprofen/oxycodone unitary oral dosage form comprising about 5 mg oxycodone hydrochloride and about 400 mg ibuprofen for the treatment of acute pain;
(b) sensitizing a doctor to a product claim that the unitary oral dosage form has a faster onset of pain relief than contemporaneous administration of a first oral dosage form comprising about 5 mg oxycodone hydrochloride and a second oral dosage form comprising about 400 mg ibuprofen;
(c) diagnosing a patient as suffering from acute pain by the doctor;
(d) selectively prescribing the ibuprofen/oxycodone unitary oral dosage form to the patient by the sensitized doctor in view of the diagnosis of the patient; and
(e) orally administering the prescribed ibuprofen/oxycodone unitary oral dosage form to the patient.

6. A method for treating a patient suffering from acute pain comprising the steps of:
(a) purchasing an ibuprofen/oxycodone unitary oral dosage form comprising about 5 mg oxycodone hydrochloride and about 400 mg ibuprofen;
(b) tendering a prescription for the patient suffering from acute pain before completing the purchase transaction, the prescription having been received in response to a diagnosis of acute pain by a doctor, the doctor (1) having been sensitized to a product claim that the unitary oral dosage form has a faster onset of pain relief than contemporaneous administration of a first oral dosage form comprising about 5 mg oxycodone hydrochloride and a second oral dosage form comprising about 400 mg ibuprofen, and (2) having selectively prescribed the unitary dosage form; and
(c) orally administering the purchased ibuprofen/oxycodone unitary oral dosage form to the patient.

7. A method of treating a patient suffering from acute pain comprising the steps of:
(a) marketing to doctors an ibuprofen/oxycodone unitary oral dosage form comprising about 5 mg oxycodone hydrochloride and about 400 mg ibuprofen for the treatment of acute pain and as exhibiting a more rapid absorption of the oxycodone hydrochloride than contemporaneous administration of a first oral dosage form comprising about 5 mg oxycodone hydrochloride and a second oral dosage form comprising about 400 mg ibuprofen;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient.

8. A method of treating a patient suffering from acute pain comprising the steps of:
(a) marketing to doctors an ibuprofen/oxycodone unitary oral dosage form comprising about 10 mg oxycodone hydrochloride and about 400 mg ibuprofen for the treatment of acute pain and as having a faster onset of pain relief than contemporaneous administration of a first oral dosage form comprising about 10 mg oxycodone hydrochloride and a second oral dosage form comprising about 400 mg ibuprofen;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient.

9. A method of treating a patient suffering from acute pain comprising the steps of:
(a) marketing to doctors an ibuprofen/oxycodone unitary oral dosage form comprising about 10 mg oxycodone hydrochloride and about 400 mg ibuprofen for the treatment of acute pain and as exhibiting a more rapid absorption of the oxycodone hydrochloride than contemporaneous administration of a first oral dosage form comprising about 10 mg oxycodone hydrochloride and a second oral dosage form comprising about 400 mg ibuprofen;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient.

10. A method of treating a patient suffering from acute pain comprising the steps of:
(a) marketing to doctors an ibuprofen/oxycodone unitary oral dosage form comprising about 5 or 10 mg oxycodone hydrochloride and about 400 mg ibuprofen for the treatment of acute pain and as providing partial or complete pain relief within 25 minutes after administration;
(b) diagnosing a patient as suffering from acute pain by a doctor;
(c) prescribing the ibuprofen/oxycodone unitary oral dosage form to the patient by the doctor in response to the marketing of the ibuprofen/oxycodone unitary oral dosage form and the diagnosis of the patient; and
(d) orally administering the prescribed ibuprofen/oxycodone unitary oral dosage form to the patient.
